(19) United States
(12) Patent Application Publication
(10) Pub. No.: US 2025/0148128 A1
(43) Pub. Date: May 8, 2025

(54) SMART BIG DE-IDENTIFICATION AND META-IZATION PROCESS

(71) Applicant: MISOINFO Tech., Seoul (KR)

(72) Inventors: Dong-wook An, Seoul (KR); Sang-do NAM, Seoul (KR); Jin-ho SON, Hanam-si (KR); Dong Woo KIM, Incheon (KR); Tae Hoon KIM, Uijeongbu-si (KR)

(21) Appl. No.: 18/545,796

(22) Filed: Dec. 19, 2023

(30) Foreign Application Priority Data
Nov. 8, 2023 (KR) 10-2023-0153292

(51) Int. Cl. G06F 21/62; G16B 50/30; G16H 30/20

(52) U.S. Cl. CPC G06F 21/6254; G16B 50/30; G16H 30/20

(57) ABSTRACT

One aspect of the present disclosure relates to a smart BIG de-identification and meta-ization process for building a data lake through meta-extraction, structured/unstructured data structuring and loading automation, which are necessary for building a CDW.

### BACKGROUND OF THE INVENTION

### 1. Field of the Invention

One aspect of the present disclosure relates to a de-identification and meta-ization process, and more particularly, to a smart BIG de-identification and meta-ization process for easily building a data lake by automatizing meta-extraction, structured/unstructured data structuring and loading, which are necessary for building a CDW.

### 2. Description of the Prior Art

In general, with regard to medical practices dealing with a patient's life, clinical diagnosis takes a large part in treating the patient, and the development of medical technology is helping to make an accurate clinical diagnosis, and its dependence will increase even more in the future.

Accordingly, although medical imaging equipment such as computer tomography (CT), magnetic resonance imaging (MRI), etc., has become essential equipment in modern medicine, medical services so far have included medical imaging equipment of photographing an abnormal part of the patient, which would be then printed into a film, and delivered to the patient's doctor, thus requiring a lot of time and manpower until a final clinical diagnosis, and resulting in inefficient resource operation without any help for a hospital's finances, and above all, the patient could not receive prompt and accurate treatment.

In addition, X-ray films are currently required to be stored in South Korea for five years, and X-ray films are classified and stored for each patient in each hospital. As the size of hospitals increases and the number of patients increases, the number of X-ray films to be stored increases. As a result, there are problems, such as waste of space, manpower and the like due to storage and management of films. For example, problems, such as a defect film having a poor storage state, re-photographing due to a loss of the film, medical disputes due to the loss of the film, and waste of time, manpower and the like required to find the stored film become serious.

Meanwhile, with the development of computer and communication technologies, systems using computer and data communication technologies have been also researched and developed in medical circles which deal with the lives of patients. As one example, a picture archiving communication system (PACS) has recently been introduced, in which a computer communication network is installed in an entire hospital, all X-ray films are converted into digital data to form a database, which will be then stored in a large storage medium connected to a server, and a desired X-ray image of a patient is viewed through a computer monitor in each clinic as necessary.

The PACS refers to a comprehensive digital image management system and a comprehensive digital image transmission system in which medical images, particularly, radiological diagnostic images, are acquired in a digital form and then transmitted over a high-speed network, and since the medical images are stored as digital data instead of past X-ray films, radiologists and clinicians give medical treatment to patients using an image inquiry device instead of the existing film view box.

The ultimate goal of the PACS is to construct a filmless hospital system, and to this end, technologies such as image display and processing, data communication and networking, a database, information management, a user interface, data storage and management and the like should be comprehensively constructed.

The communication in the PACS is based on a digital imaging and communications in medicine (DICOM) protocol as a standard, and the DICOM protocol refers to a communication protocol which effectively supports communication between various digital image acquisition devices such as nuclear medicine, ultrasound, and the like, as well as CT and MRI, and other information systems using an industry standard network, and the first standard was established in 1985, the following year, in which the American College of Radiology (ACR) and the National Electrical Manufacturers Association (NEMA) jointly started standardization in 1984.

After that, the standard has reached the current 3.0 revision through two revision works in 1988 and 1993, and is finally called DICOM.

The reason why the DICOM protocol has emerged is because, as the medical industry has been informed, each medical equipment is often used in connection with each other rather than being independently used, and certain promises are needed in exchanging medical images and related information between medical imaging equipment.

In other words, in the past, there was no predetermined standard, and a method of storing and communicating information varied according to manufacturers, types of imaging equipment, or models of imaging equipment, and thus, an expensive gateway needed to be purchased in order to exchange information with each other, or communication could not be performed at all.

However, with the establishment of the DICOM standard, equipment which conforms to this standard may exchange information with each other regardless of manufacturer and equipment type, even without the need for a special converter. This is not limited only to communication between DICOM support equipment present in a hospital, but suggesting that communication with remote locations is also possible.

In addition, since a network configuration method also follows a standard method widely used in the computer industry at present, the DICOM standard may be easily applied to all medical image-related systems including connection between hospital centers, communication between remote clinics, and even a remote diagnosis system. The PACS system adopting the DICOM standard stores medical image data obtained by photographing a patient, that is, a subject, and also stores medical image data obtained by different apparatuses, for example, CT, MRI, etc.

Meanwhile, after in-hospital X-Ray, MRI, and CT imaging, the data are temporarily located in a file server before being transmitted to the PACS system. In recent years, there has been an increasing movement to build a data lake by extracting meta from files located on the file server, structuring structured/unstructured data, and automating loading. However, there is still a lack of a process capable of smoothly performing such a series of processes, thus a research thereon is being conducted.

### RELATED ART REFERENCES

### Patent Documents

(Patent Document 0001) Korean Registered Patent Publication No. 10-0696708 (publicized on Mar. 20, 2007), “Online Transmission System of Medical Information Between Medical Institutions”

### National Research and Development Project for Supporting Present Disclosure

| | |
|---|---|
| [Task ID No.] | 1711195670 |
| [Task No.] | 00223657 |
| [Ministry name] | Ministry of Science and ICT |
| [Task management | Institute of Information Communications |
| (specialized) agency | Technology Planning & Evaluation |
| Name] | |
| [Research project | Digital conversion K-SW technology |
| name] | development (R&D) |
| [Research task name] | Clinical Data Research Analysis and Artificial |
| | Intelligence Platform Development Based on |
| | Closed Cloud for Medical Data Research |
| [Contribution rate] | 1/1 |
| [Task execution agency | MISOInfo Tech. |
| name] | |
| [Research period] | Apr. 1, 2023~Dec. 31, 2024 |

### SUMMARY OF THE INVENTION

In order to solve the above problems, an object of one aspect of the present disclosure is to provide a smart BIG de-identification and meta-ization process for easily building a data lake by automatizing meta-extraction, structured/unstructured data structuring and loading, which are necessary for building a CDW.

In order to achieve the above object, one aspect of the present disclosure may provide a smart BIG de-identification and meta-ization process which includes: an event detection and source data acquisition step S10 of detecting an event from a file server in which clinical data is stored and obtaining a source data file; a meta extraction step S20 of extracting file meta from the source data file according to a preset meta format; a de-identification step S30 of de-identifying unnecessary meta from the source data file to form a processed data file; a meta-based structure generation step S40 of generating an object address standardized according to a data structure preset by a user; a meta and object address indexing step S50 of indexing the standardized object address and the extracted meta to a database management system; and a database loading step S60 of loading the processed data file on an object-based storage in a data lake as the standardized object address.

Meanwhile, in the event detection and source acquisition step S10, the source data file may be obtained from the file server in which clinical data is stored through a WatchServicer-based event detection client or an ansible-based client.

Meanwhile, in the meta extraction step S20, the file meta may be extracted from a Dicom header according to a preset meta format using a Dicom-related library including pydicom and dcm4che from the source data file.

Meanwhile, in the meta-based structure generation step S40, the standardized object address may be generated using a meta+file name or mapping clinical data extracted according to a data structure preset by the user.

Meanwhile, the meta-based structure generation step S40; the meta and object address indexing step S50; and the database loading step S60 may be performed through a data warehouse.

A smart BIG de-identification and meta-ization process according to one aspect of the present disclosure can easily build a data lake by automatizing meta-extraction, structured/unstructured data structuring and loading, which are necessary for building a CDW.

### BRIEF DESCRIPTION OF THE DRAWINGS

FIG. 1 is an illustrative view showing a system for performing a method of verifying a quality of data in accordance with an embodiment of the present disclosure.

FIG. 2 is a flowchart of a smart BIG de-identification and meta-ization process according to an embodiment of the present disclosure and a relationship with a surrounding configuration.

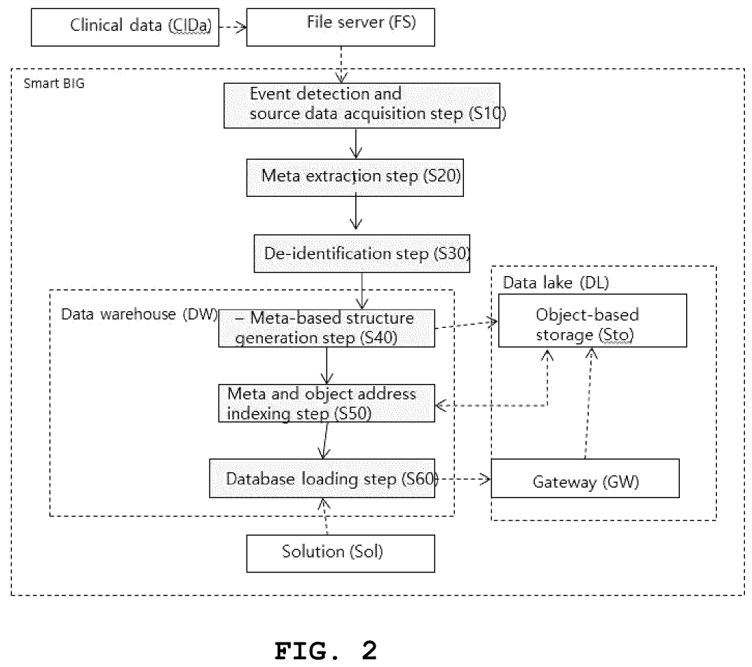

FIG. 3 is a general schematic view showing an exemplary computing environment in which embodiments of the present disclosure may be implemented.

### DETAILED DESCRIPTION OF THE INVENTION

Hereinafter, various embodiments and/or aspects will be disclosed with reference to drawings. In the following description, multiple concrete details will be disclosed in order to help general understanding of one or more aspects for the purpose of description. However, it will be recognized by those skilled in the art that the aspect(s) can be executed without the concrete details. In the following description and accompanying drawings, specific exemplary aspects of one or more aspects will be described in detail. However, these aspects are illustrative and some of the various methods in the principles of the various aspects may be utilized, and the described descriptions are intended to include all such aspects and their equivalents. Specifically, it is not intended that any “embodiment,” “example,” “aspect,” “illustration” and the like used in the present specification are preferable or advantageous over any other aspects or designs.

Hereinafter, the same reference numerals are assigned to the same or similar elements regardless of reference numerals in the drawings, and redundant descriptions thereof will be omitted. Further, in the description of the embodiments disclosed in the present specification, a detailed description of related known technology will be omitted when it may make the subject matter of the embodiments disclosed in the present specification unnecessarily unclear. In addition, the accompanying drawings are only for easily understanding the embodiments disclosed in the present specification, and the technical spirit disclosed in the present specification is not limited by the accompanying drawings.

Although the first, second, etc. are used to describe various elements or components, these elements or components are not limited by these terms. These terms are only used to distinguish one element or component from another element or component. Accordingly, a first element or component mentioned below may be a second element or component within the technical spirit of the present disclosure.

All terms (including technical and scientific terms) used herein may be used with the meaning commonly understood by those skilled in the art to which the present disclosure pertains, unless otherwise defined. In addition, terms defined in a commonly used dictionary are not to be interpreted ideally or excessively unless clearly defined in particular.

Further, the term “or” is intended to mean an inclusive “or” rather than an exclusive “or.” In other words, unless otherwise specified or if not clear in the context, “X uses A or B” is intended to mean one of the natural inclusive substitutions. In other words, if X uses A; X uses B; or X uses both A and B, “X uses A or B” may be applied in either of these cases. In addition, it needs to be understood that the term “and/or” used herein refers to and includes all possible combinations of one or more of the listed related items.

Further, the terms “includes” and/or “including” may mean that a corresponding feature and/or component exists, but it should be understood that the terms “include” and/or “including” do not exclude presence or addition of one or more other features, components, and/or a group thereof. Moreover, unless otherwise specified or if it is not clear in the context to indicate a singular form, in the present specification and claims, the singular needs to be generally interpreted to mean “one or more.”

In addition, the terms “information” and “data” as used herein may often be used interchangeably with each other.

When one element is described as being “connected” or “accessed” to another element, it shall be construed as being connected or accessed to the other element directly but also as possibly having another element in between. On the contrary, if one element is described as being “directly connected” or “directly accessed” to another element, it shall be construed that there is no other element in between.

The suffixes “module” and “unit” for the components used in the following description are given or mixed in consideration of only the ease of writing the specification, and do not have meanings or roles distinguished from each other by themselves.

Objects and effects of the present disclosure and technical configurations for achieving the same will become apparent with reference to the embodiments described below in detail along with the accompanying drawings. In the following description of the present disclosure, a detailed description of known functions and configurations will be omitted when it may make the subject matter of the present disclosure unnecessarily unclear. Further, terms to be described below are terms defined in consideration of functions in the present disclosure, and may vary according to the intention or custom of a user or an operator.

However, the present disclosure may be implemented in various different forms without limitation to the embodiments disclosed below. The present embodiments are provided only to make the present disclosure complete and to fully inform those skilled in the art to which the present disclosure pertains of the scope of the disclosure, and the present disclosure is only defined by the scope of the claims. Thus, the definition needs to be made based on the contents throughout this specification.

In the present disclosure, the computing device may provide a function such as an interface to perform a smart BIG de-identification and meta-ization process according to a request received from the user terminal. Specifically, the computing device may invoke an automated learning model to perform the smart BIG de-identification and meta-ization process. The automated learning model may be an interface made to control functions provided by an operating system or a programming language. The automated learning model may assist in interfacing or working from the input data through a pre-trained model, and may return a result according to the input to the computing device. In addition, the computing device may transmit a result according to the input to a user terminal.

Hereinafter, the smart BIG de-identification and meta-ization process according to one aspect of the present disclosure will be described in detail with reference to the accompanying drawings.

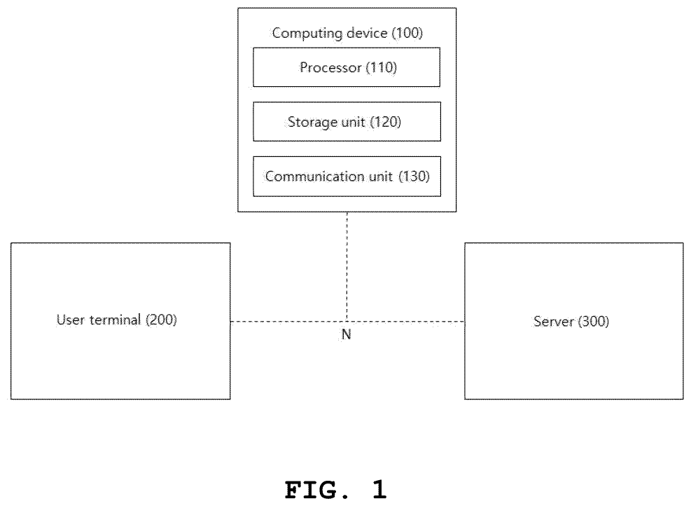

FIG. 1 shows an exemplary basic system for performing a smart BIG de-identification and meta-ization process according to some embodiments of the present disclosure.

Referring to FIG. 1, the system for performing the smart BIG de-identification and meta-ization process may include a computing device 100, a user terminal 200, a server 300, and a network N. However, the above-described components are not essential in implementing the smart BIG de-identification and meta-ization process, and thus the smart BIG de-identification and meta-ization process may have more or fewer components than those listed above.

The computing device 100 may include any type of computer system or computer device such as, for example, a microprocessor, a mainframe computer, a digital processor, a portable device, a device controller, or the like.

The processor 110 may conventionally process an overall operation of the computing device 100. The processor 110 may process signals, data, information, and the like input or output through the components included in the computing device 100 or drive an application program stored in a storage unit 120, thereby providing or processing information or functions appropriate for a user.

In one aspect of the present disclosure, the processor 110 may request clinical data from the server 300 as a request for performing the smart BIG de-identification and meta-ization process is received from the user terminal 200. Here, the smart BIG de-identification and meta-ization execution information may be an interface which is made to control functions provided by an operating system or a programming language. As one example, the smart BIG de-identification and meta-ization process execution information may build a data lake through a pre-trained model. The smart BIG de-identification and meta-ization process execution information may be information for the processor 110 to perform the smart BIG de-identification and meta-ization process. According to an embodiment, the smart BIG de-identification and meta-ization execution information may include at least one of an internet protocol (IP) address, a host address, and port information. The processor 110 may assist in building a data lake based on the smart BIG de-identification and meta-ization process.

The storage unit 120 may include a memory and/or a permanent storage medium. The memory may include at least one storage medium of a flash memory type, a hard disk type, a multimedia card micro type, a card type memory (for example, an SD, XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk.

A communication unit 130 may include one or more modules which enable communication between the computing device 100 and a communication system, between the computing device 100 and the user terminal 200, between the computing device 100 and the server 300, or between the computing device 100 and the network N. The communication unit 130 may include at least one of a mobile communication module, a wired internet module, and a wireless internet module.

The user terminal 200 may include a personal computer (PC), a notebook, a mobile terminal, a smart phone, a tablet PC, and the like owned by a user, and may include all types of terminals capable of connecting to a wired/wireless network.

In one aspect of the present disclosure, the user terminal 200 may receive a request for performing the smart BIG de-identification and meta-ization process from the user. The user terminal 200 may receive a series of interfaces, etc., by transmitting the smart BIG de-identification and meta-ization process execution request information to the computing device 100. As a result is received, the user terminal 200 may display the received result.

According to some embodiments of the present disclosure, the computing device 100 and the user terminal 200 may be implemented as one configuration. In other words, the computing device 100 may be the user terminal 200, and the user terminal 200 may be the computing device 100. This will be differently applied depending on a use environment.

The server 300 may include any type of computer system or computer device such as, for example, a microprocessor, a mainframe computer, a digital processor, a portable device, a device controller, and the like.

According to some embodiments of the present disclosure, the server 300 may store clinical data. As one example, the clinical data may be dynamically expanded. Here, a phrase “dynamically expanded” may mean that it is automatically generated/added without an administrator's intervention. For example, the clinical data may be further generated based on an external input. The additionally generated clinical data may be stored in the server 300. Thus, the server 300 may store connection information for all clinical data.

Alternatively, according to some embodiments of the present disclosure, a data lake for clinical data may be built in the server 300. The user may perform the smart BIG de-identification and meta-ization process to build the data lake and store the same in the server 300.

In addition, according to some embodiments of the present disclosure, a server for providing clinical data and a server for building the data lake may exist separately. In other words, according to an embodiment of the present disclosure, a plurality of servers 300 may be provided.

According to some embodiments of the present disclosure, the computing device 100 and the server 300 may be implemented as one entity. Accordingly, the computing device 100 and the server 300 may be implemented as one configuration. For example, the computing device 100 may be included in the server 300 to operate as one configuration.

The network N may be configured regardless of a communication aspect thereof such as wired, wireless and the like, and may be configured as various communication networks such as a personal area network (PAN), a wide area network (WAN) etc. In addition, the network may be a known world wide web (WWW), and may use a wireless transmission technology used in short range communication such as infrared data association (IrDA) or Bluetooth. The techniques described in the present specification may be used in the networks mentioned above, and may be also used in other networks.

The smart BIG de-identification and meta-ization process according to one aspect of the present disclosure may include: an event detection and source data acquisition step S10 of detecting an event from a file server FS in which clinical data ClDa is stored and obtaining a source data file; a meta extraction step S20 of extracting file meta from the source data file according to a preset meta format; a de-identification step S30 of de-identifying unnecessary meta from the source data file to form a processed data file; a meta-based structure generation step S40 of generating an object address standardized according to a data structure preset by a user; a meta and object address indexing step S50 of indexing the standardized object address and the extracted meta to a database management system DBMS; and a database loading step S60 of loading the processed data file on an object-based storage Sto in a data lake DL as the standardized object address.

In the event detection and source data acquisition step S10, an event may be detected and a source data file may be obtained from a file server FS in which clinical data ClDa is stored.

More specifically, in the event detection and source data acquisition step S10, the source data file may be obtained from the file server FS in which clinical data ClDa is stored through a WatchServicer-based event detection client or an ansible-based client. In the event detection and source data acquisition step S10, the source data file obtained may be a Dicom-related file.

The WatchServicer may detect a change in a file or directory registered with a separate thread and return the detected change to as event. It may be useful for notification of various tasks, such as security monitoring, property file changes and the like. It may detect when a file is created, deleted, or modified in a specific directory.

The ansible may be an open-source software provisioning, configuration management, and application deployment tool. It may run on numerous Unix-based systems and allow the configuration of Unix-based operating systems and Microsoft windows. It may include a self-declarative language to describe the system configuration.

The data obtained as above may have meta extracted through the meta extraction step S20.

In the meta extraction step S20, a file meta may be extracted according to a preset meta format from the source data file obtained in the event detection and source data acquisition step S10.

More specifically, in the meta extraction step S20, the file meta may be extracted from a Dicom header according to a preset meta format using a Dicom-related library including pydicom and dcm4che from the source data file.

The pydicom may be a package used when handling a Dicom file (.dcm) in the Python language. The pydicom may read, modify and write DICIOM data in a pure Python package for working with DICOM files. dcm4che may be a collection of open source applications and utilities for medical companies. dcm4che was developed as the Java programming language for performance and portability.

File meta in one aspect of the present disclosure may refer to metadata recorded in the DICOM header. The DICOM header may include standard data elements. The standard data element may refer to elements related to a medical image defined by the DICOM standard. For example, a medical practitioner may look at a value of an attribute “BodyPartExamined” of the DICOM header to determine whether a medical image is a body part of a patient to be read, and proceed with medical image reading. Furthermore, the medical practitioner may normalize original images coming from various environments by using the “Window Center/Width” attribute of the DICOM header. In addition, the DICOM header may include a non-standard data element. The non-standard data element may refer to an element related to a medical image, which is not defined according to the DICOM standard, but is generated according to the needs of a medical imaging apparatus manufacturer or a medical institution. For example, the file meta may include at least one of medical image creation time information, photographing place information, photographing equipment information, or information on an administrator who created the medical image, the creation time information may represent a time when the medical image is created, and the photographing place information may include at least one of an address of a place where the medical image was created or a name of the place.

In the meta extraction step S20, the meta may be extracted using a meta format preset by the user so that only necessary file meta may be extracted from the source data file.

In the de-identification step S30, unnecessary meta may be de-identified from the source data file to form a processed data file.

In the de-identification step S30, the unnecessary meta in the source data file may be information which the user does not need. One or more of unnecessary information and location information of the unnecessary information may be obtained, respectively and a data file processed based on the unnecessary information and the location information of the unnecessary information may be formed. Alternatively, in the de-identification step S30, the unnecessary meta in the source data file may be sensitive information which is personal information of the patient. One or more of sensitive information and location information of the sensitive information may be obtained, and a data file processed based on the sensitive information and the location information may be formed.

The data processed in the de-identification step S30 may be data processed by at least one method of changing at least a part of unnecessary information or sensitive information or processing the same with a de-identification filter. Examples of the de-identification processing may be performed in a manner consistent with mosaic, transparency, and ambient color. Alternatively, a method of deleting the unnecessary meta per se may be used.

After the de-identification step S30 is performed, a processed data file may be obtained.

In the meta-based structure generation step S40, the standardized object address may be generated according to a data structure preset by the user.

More specifically, in the meta-based structure generation step S40, the standardized object address may be generated using a meta+file name or mapping clinical data extracted according to a data structure preset by the user.

The standardized object address in the meta-based structure generation step S40 may be intended to set an address stored in the data lake DL by assigning a meta+file name or to set an address stored in the object-based storage Sto of the data lake DL using mapping clinical data.

The meta-based structure generation step S40 may be performed in relation to a data warehouse DW. The data warehouse may refer to a database which converts and manages data accumulated in a database of a backbone system into a common format to help users make a decision. For short, the data warehouse may be also called DW. In summary, the data warehouse may be a database containing data necessary for user decision-making. The meta-based structure generation step S40 may be performed through the data warehouse to enable data-based decision-making, integrate and analyze data of several sources, analyze past data, and have an advantage of providing better information using existing information.

In the meta and object address indexing step S50, the standardized object address and the extracted meta may be indexed in the database management system (DBMS).

Here, the database management system may be an application program which provides functions for creating, storing, and managing a set of data called a database. In other words, the database management system may be a program specialized for data management. Depending on types, the database management system may support up to DataBase Server. The database management system may be software for operating and managing a database, and a database in which various data are stored may be shared with several users or application programs and simultaneously accessed.

In the meta and object address indexing step S50, indexing of the standardized object address and the extracted meta in the database management system (DBMS) may be intended to load the data file processed in the database loading step S60, which will be described later, on the object-based storage Sto in the data lake DL as the standardized object address.

The meta and object address indexing step S50 may be performed in connection with the data warehouse DW. The data warehouse may refer to a database which converts and manages data accumulated in a database of a backbone system into a common format to help users make a decision. For short, the data warehouse may be also called DW. In summary, the data warehouse may be a database containing data necessary for user decision-making. The meta and object address indexing step S50 may be performed through the data warehouse to enable data-based decision-making, integrate and analyze data of several sources, analyze past data, and have an advantage of providing better information using existing information.

In the database loading step S60, the processed data file may be loaded on the object-based storage Sto in the data lake DL as the standardized object address.

More specifically, in the database loading step S60, the standardized object address and the extracted meta from the meta and object address indexing step S50 may be indexed in the database management system (DBMS), while the processed data file may be loaded on the object-based storage Sto in the data lake DL as the standardized object address at the same time. Meanwhile, the database loading step S60 may pass through a gateway GW prior to loading on the object-based storage Sto as the standardized object address.

In other words, the database loading step S60 may be simultaneously performed with the meta and object address indexing step S50.

The database loading step S60 may be performed in relation to a data warehouse DW. The data warehouse may refer to a database which converts and manages data accumulated in a database of a backbone system into a common format to help users make a decision. For short, the data warehouse may be also called DW. In summary, the data warehouse may be a database containing data necessary for user decision-making. The database loading step S60 may be performed through the data warehouse to enable data-based decision-making, integrate and analyze data of several sources, analyze past data, and have an advantage of providing better information using existing information.

Meanwhile, the meta-based structure generation step S40; the meta and object address indexing step S50; and the database loading step S60 may be performed while interacting with the data lake DL. In other words, not only in the database loading step S60, data may be loaded, but also in the meta-based structure generation step S40; and the meta and object address indexing step S50, all processes of transmitting and receiving data to and from the data lake DL, setting the standardized object address, indexing the standardized object address and the extracted meta, and storing the database may be performed in association with the data lake DL, thereby minimizing problems which may occur in the loading process.

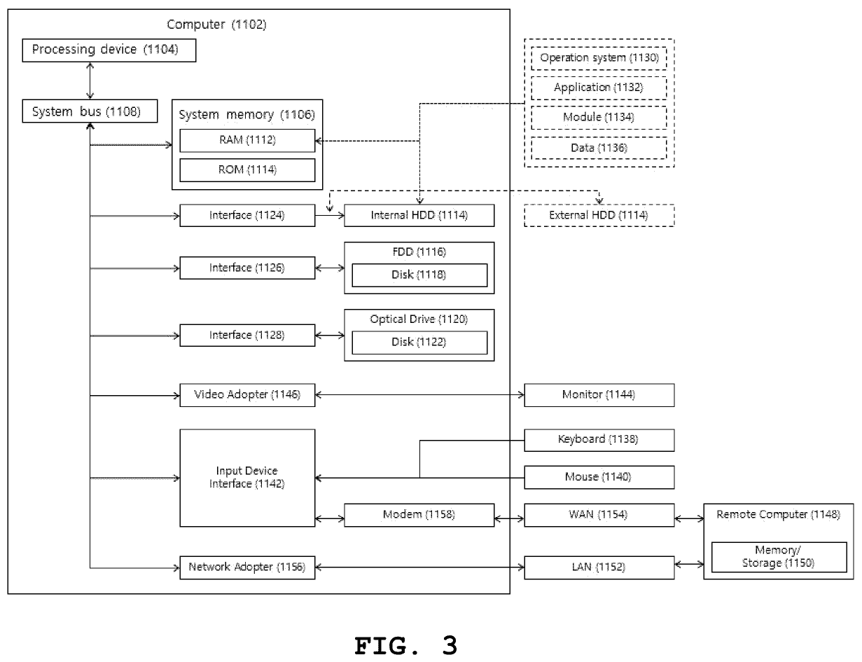

FIG. 3 is a general schematic view showing an exemplary computing environment in which embodiments of the present disclosure may be implemented.

While the present disclosure has been generally described above in connection with computer-executable instructions which may be executed on one or more computers, those skilled in the art will appreciate that the present disclosure may be implemented in combination with other program modules and/or as a combination of hardware and software.

In general, modules in the present specification may include routines, procedures, programs, components, data structures, and the like which perform particular tasks or implement particular abstract data types. Those skilled in the art will also appreciate that the methods of the present disclosure may be practiced with other computer system configurations, including single-processor or multi-processor computer systems, mini-computers, mainframe computers, as well as personal computers, hand-held computing devices, microprocessor-based or programmable consumer electronics, and the like, each of which may operate in conjunction with one or more associated devices.

The described embodiments of the present disclosure may be also practiced in a distributed computing environment where certain tasks are performed by remote processing devices which are connected through a communication network. In the distributed computing environment, program modules may be located in both local and remote memory storage devices.

A computer may typically include a variety of computer-readable media. Media accessible by a computer may include volatile and nonvolatile media, transitory and non-transitory media, removable and non-removable media. By way of example, and not limitation, the computer-readable media may include computer-readable storage media and computer-readable transmission media.

Computer-readable storage media may include volatile and non-volatile media, transitory and non-transitory media, removable and non-removable media implemented in any method or technology which stores information such as computer-readable instructions, data structures, program modules, or other data. Computer-readable storage media may include, but is not limited to, RAM, ROM, EEPROM, flash memory or other memory technology, CD-ROM, digital video disks (DVD) or other optical disk storage, magnetic cassette, magnetic tape, magnetic disk storage or other magnetic storage devices, or any other media which may be accessed by a computer and used to store the desired information.

The computer-readable media may conventionally implement computer-readable instructions, data structures, program modules, other data, or the like in a modulated data signal such as a carrier wave or other transport mechanisms, and may include all information transmission media. The term “modulated data signal” may refer to a signal which has set or changed one or more of the properties of that signal to encode information within the signal. By way of example, and not limitation, computer-readable transmission media may include wired media such as a wired network or direct-wired connection, and wireless media such as acoustic, RF, infrared and other wireless media. A combination of any of the media described above may be also intended to be included within the scope of computer-readable transmission media.

An exemplary environment 1100 including a computer 1102 and implementing various aspects of the present disclosure may be provided, and the computer 1102 may include a processing device 1104, a system memory 1106 and a system bus 1108. The system bus 1108 may connect system components including the system memory 1106 (not limited thereto) to the processing device 1104. The processing device 1104 may be any processor among various commercial processors. A dual processor and other multiprocessor architectures may also be used as the processing device 1104.

The system bus 1108 may be any of several types of bus structures which may be additionally interconnected to a local bus using any of a memory bus, a peripheral device bus, and various commercial bus architectures. The system memory 1106 may include read only memory (ROM) 1110 and random access memory (RAM) 1112. A basic input/output system (BIOS) may be stored in a non-volatile memory 1110, such as ROM, EPROM, EEPROM, etc., in which the BIOS may include basic routines which help to transfer information between components within the computer 1102, such as during start-up. The RAM 1112 may also include a high-speed RAM such as a static RAM, etc., for caching data.

The computer 1102 may also include an internal hard disk drive (HDD) 1114 (e.g., EIDE, SATA), in which the internal hard disk drive 1114 may be also configured for external use within a suitable chassis (not shown), a magnetic floppy disk drive (FDD) 1116 (e.g., for reading from or writing to a removable diskette 1118), and an optical disk drive 1120 (e.g., for reading a CD-ROM disk 1122 or for reading from or writing to other high capacity optical media such as the DVD, etc.). The hard disk drive 1114, the magnetic disk drive 1116, and the optical disk drive 1120 may be connected to the system bus 1108 by a hard disk drive interface 1124, a magnetic disk drive interface 1126, and an optical drive interface 1128, respectively. The interface 1124 for implementing an exterior drive may include, for example, at least one or both of a universal serial bus (USB) and an IEEE 1394 interface technology.

These drives and their associated computer-readable media may provide non-volatile storage of data, data structures, computer-executable instructions, and the like. In the case of the computer 1102, the drive and media may correspond to storing any data in an appropriate digital format. Although the description of the computer-readable storage media above refers to a HDD, a removable magnetic disk, and a removable optical media such as a CD, DVD or the like, it will be appreciated by those skilled in the art that other types of storage media readable by a computer, such as a zip drive, magnetic cassette, flash memory card, cartridge, and the like, may be also used in the exemplary operating environment, and any such media may include computer-executable instructions for performing the methods of the present disclosure.

A plurality of program modules including an operating system 1130, one or more application programs 1132, other program modules 1134, and program data 1136 may be stored in the drive and the RAM 1112. All or portions of the operating system, applications, modules, and/or data may be also cached in the RAM 1112. It will be appreciated that the present disclosure may be implemented in a variety of commercially available operating systems or combinations of operating systems.

A user may enter commands and information into the computer 1102 through one or more wire/wireless input devices, for example, a pointing device, such as a keyboard 1138, a mouse 1140, and the like. Other input devices (not shown) may include a microphone, an IR remote controller, a joystick, a game pad, a stylus pen, a touch screen, and the like. These and other input devices may be often connected to the processing unit 1104 through an input device interface 1142 which is coupled to the system bus 1108, but may be connected by other interfaces, such as a parallel port, an IEEE 1394 serial port, a game port, a USB port, an IR interface, and the like.

A monitor 1144 or other types of display device may be also connected to the system bus 1108 via an interface, such as a video adapter 1146, etc. In addition to the monitor 1144, computers may generally include other peripheral output devices (not shown), such as speakers, printers, and the like.

The computer 1102 may operate in a networked environment using logical connections via wired and/or wireless communications to one or more remote computers, such as a remote computer(s) 1148. The remote computer(s) 1148 may be a workstation, a server computer, a router, a personal computer, a portable computer, a microprocessor-based entertainment appliance, a peer device or other conventional network nodes, and may generally include many or all of the elements described with regard to the computer 1102, although, for purposes of brevity, only a memory storage device 1150 is illustrated. The illustrated logical connections may include wired/wireless connections to a local area network (LAN) 1152 and/or a larger network, for example, a wide area network (WAN) 1154. The LAN and WAN networking environments may be general environments in offices and companies, and facilitate an enterprise-wide computer network such as an intranet, and the like, all of which may be connected to a worldwide computer network, for example, the Internet.

When used in a LAN networking environment, the computer 1102 may be connected to the local network 1152 through a wired and/or wireless communication network interface or adapter 1156. The adapter 1156 may facilitate wired or wireless communications to the LAN 1152, and the LAN 1152 may also include a wireless access point installed thereon for communicating with the wireless adapter 1156. When used in a WAN networking environment, the computer 1102 may include a modem 1158, or may be connected to a communications server on the WAN 1154, or may have other means for establishing communications over the WAN 1154, such as by way of the Internet. The modem 1158, which may be internal or external and may be a wired or wireless device, may be connected to the system bus 1108 through the serial port interface 1142. In the networked environment, the program modules described for the computer 1102 or some thereof may be stored in the remote memory/storage device 1150. It will be appreciated that the illustrated network connection may be exemplary and other means of establishing a communication link between computers may be used.

The computer 1102 may be operative to communicate with any wireless device or entity deployed and operating in wireless communication, for example, a printer, scanner, desktop and/or portable computer, portable data assistant (PDA), communication satellite, any equipment or location associated with a wireless detectable tag, and telephone. This may include at least Wi-Fi and Bluetooth radio technology. Thus, the communication may be a predefined structure as in a conventional network or simply an ad hoc communication between at least two devices.

Wireless Fidelity (Wi-Fi) may enable connection to the Internet or the like without wires. Wi-Fi may be a wireless technology, such as a cell phone, which allows such devices, e.g., computers, to send and receive data indoors and outdoors, i.e., anywhere within the coverage area of a base station. The Wi-Fi network may use a wireless technology called IEEE 802.11 (a, b, g, and others) in order to provide safe, reliable, and high-speed wireless connection. Wi-Fi may be used to connect computers to each other, to the Internet, and to wired networks (using IEEE 802.3 or Ethernet). The Wi-Fi network may operate, for example, at a data rate of 11 Mbps (802.11a) or 54 Mbps (802.11b) in unlicensed 2.4 and 5 GHz wireless bands or may operate in a product including both bands (dual bands).

Those of ordinary skill in the art of the present disclosure will appreciate that the various illustrative logical blocks, modules, processors, means, circuits, and algorithm steps described in connection with the embodiments disclosed herein may be implemented by electronic hardware, various forms of program or design code (referred to herein, for convenience, as “software”), or a combination of all. To clearly describe this interchangeability of hardware and software, various illustrative components, blocks, modules, circuits, and steps have been generally described above in relation to their functionality. Whether this functionality is implemented as hardware or software may depend on design constraints imposed on a particular application and an overall system. Those of ordinary skill in the art of the present disclosure may implement the described functions in various ways for each specific application, but such implementation decisions should not be interpreted as being out of the scope of the present disclosure.

Various embodiments presented herein may be implemented in a method, apparatus, or article of manufacture using standard programming and/or engineering techniques. The term “article of manufacture” may include a computer program or media accessible from any computer-readable device. For example, the computer-readable storage medium may include, but is not limited to, a magnetic storage device (e.g., a hard disk, a floppy disk, a magnetic strip, etc.), an optical disk (e.g., a CD, a DVD, etc.), a smart card, and a flash memory device (e.g., a EEPROM, a card, a stick, a key drive, etc.). The term “machine-readable medium” may include, but is not limited to, a wireless channel and various other media capable of storing, retaining, and/or carrying instruction(s) and/or data.

The description of the embodiments presented may be provided so that those of ordinary skill in the art of any present disclosure may use or practice the present disclosure. Various modifications to these embodiments will be apparent to those skilled in the art of the present disclosure, and the general principles defined herein may be applied to other embodiments without departing from the scope of the present disclosure. Thus, the present disclosure may not be limited to the embodiments presented herein, but may need to be construed in the broadest range consistent with the principles and novel features presented herein.

What is claimed is:

1. A smart BIG de-identification and meta-ization process comprising:
an event detection and source data acquisition step (S10) of detecting an event from a file server in which clinical data is stored and obtaining a source data file;
a meta extraction step (S20) of extracting file meta from the source data file according to a preset meta format;
a de-identification step (S30) of de-identifying unnecessary meta from the source data file to form a processed data file;
a meta-based structure generation step (S40) of generating an object address standardized according to a data structure preset by a user;
a meta and object address indexing step (S50) of indexing the standardized object address and the extracted meta to a database management system; and
a database loading step (S60) of loading the processed data file on an object-based storage in a data lake as the standardized object address.

2. The smart BIG de-identification and meta-ization process of claim 1, wherein in the event detection and source data acquisition step (S10), the source data file is obtained from the file server in which clinical data is stored through a WatchServicer-based event detection client or an ansible-based client.

3. The smart BIG de-identification and meta-ization process of claim 1, wherein in the meta extraction step (S20), the file meta is extracted from a Dicom header according to a preset meta format using a Dicom-related library including pydicom and dcm4che from the source data file.

4. The smart BIG de-identification and meta-ization process of claim 1, wherein in the meta-based structure generation step (S40), the standardized object address is generated using a meta+file name or mapping clinical data extracted according to a data structure preset by the user.

5. The smart BIG de-identification and meta-ization process of claim 1, wherein the meta-based structure generation step (S40); the meta and object address indexing step (S50); and the database loading step (S60) are performed through a data warehouse.
